(19) United States
(12) Patent Application Publication
(10) Pub. No.: US 2025/0186334 A1
(43) Pub. Date: Jun. 12, 2025

(54) TOPICAL OCULAR THERAPY

(71) Applicant: Moxicaine, LLC, Jeffersonville, IN (US)

(72) Inventors: Erich J. BERG, Jeffersonville, IN (US); David R. HARDTEN, Bloomington, MN (US)

(73) Assignee: Moxicaine, LLC, Jeffersonville, IN (US)

(21) Appl. No.: 18/629,882

(22) Filed: Apr. 8, 2024

Related U.S. Application Data
(63) Continuation-in-part of application No. 18/537,780, filed on Dec. 12, 2023.

(51) Int. Cl. A61K 9/00; A61K 31/136; A61K 31/4709

(52) U.S. Cl. CPC A61K 9/0048; A61K 31/136; A61K 31/4709

(57) ABSTRACT

A novel ophthalmic treatment for ocular pain, such as epithelial defects of the cornea or conjunctiva, which includes a liquid or gel-based formulation containing a topical anesthetic mixed with an antibiotic. The two substances are combined according to a preferred volumetric ratio wherein the anesthetic retains its efficacy in alleviating the patient's pain, while also diluting the anesthetic enough to permit patients to self-administer the eyedrops without clinical supervision. In a preferred example, the antibiotic comprises moxifloxacin (or an equivalent fluoroquinolone-class antibiotic) at an initial concentration of about 0.5%, and the anesthetic comprises proparacaine hydrochloride at an initial concentration of about 0.5%. Preferably, the volumetric ratio of the antibiotic and an excipient relative to the anesthetic is between about 5:1 and about 15:1, such that the antibiotic and the excipient dilute the anesthetic down to a relative concentration of about 0.030% to about 0.085%.

### RELATED APPLICATIONS

The present application is a continuation-in-part of U.S. patent application Ser. No. 18/537,780, filed on Dec. 12, 2023 and entitled “CORNEAL THERAPY,” the entire contents of which are hereby incorporated by reference.

### TECHNICAL FIELD

The present technology relates to ophthalmology, optometry, and emergency medicine.

### BACKGROUND

The cornea is the transparent front layer of the human eyeball. Richly innervated by sensory nerves, this tissue is responsible for bending light to focus it onto the retina. The epithelium is the cell layer lining the corneal surface, and is continuous with the remainder of the external tissue of the eye termed the conjunctiva. Presently, accidental abrasions to the corneal and conjunctival epithelium are the most common eye-related injuries presenting to emergency rooms, accounting for around 30% of eye-related complaints. The conjunctiva is also intentionally harmed during ocular surgery as a means to access the anterior and posterior intraocular chambers, extraocular muscles, or surrounding tissues. Full-thickness abrasions, or open globe lacerations, are exquisitely painful and represent the most serious injury in eye care.

### SUMMARY OF THE DISCLOSURE

In general, the present disclosure relates to the topical treatment of ocular pain or discomfort.

In some examples, a medicated eyedrop is configured to treat ocular pain, wherein the eyedrop includes: (1) an antibiotic defining a first concentration of about 0.415% to about 0.470% of the eyedrop; (2) a topical anesthetic defining a second concentration of about 0.030% to about 0.085% of the eyedrop; and (3) an inactive portion defining a third concentration of about 99.3% to about 99.7% of the eyedrop.

In further examples, a method of forming a topical ocular therapeutic includes: (1) providing an antibiotic; (2) providing a topical anesthetic; (3) combining the antibiotic with the anesthetic according to a predetermined volumetric ratio of the antibiotic relative to the anesthetic, wherein the volumetric ratio is between about 5:1 and about 15:1.

In further examples, a method of treating a patient's ocular pain includes: (1) dispensing a medicated eyedrop from a reservoir; and (2) applying the medicated eyedrop to the eye of the patient, wherein the medicated eyedrop includes: (a) an antibiotic defining a first concentration of about 0.415% to about 0.470% of the eyedrop; (b) a topical anesthetic defining a second concentration of about 0.030% to about 0.085% of the eyedrop; and (c) an inactive portion defining a third concentration of about 99.3% to about 99.7% of the eyedrop.

Other objects, advantages, features, properties and relationships of the invention will be obtained from the following detailed description and accompanying drawings which set forth illustrative embodiments that are indicative of the various ways in which the principles of the invention may be employed.

### BRIEF DESCRIPTION OF THE DRAWINGS

FIG. 1 shows an example of a corneal epithelial abrasion, as diagnosed with fluorescent-yellow dye and a cobalt-blue filter, which illuminates the dye in the scratch on the corneal surface as bright green.

FIG. 2 shows an example of a 15-milliliter (mL) bottle of proparacaine, typical of what is used in clinics throughout the United States to diagnose ocular epithelial abrasions.

FIG. 3 is an example system for making a topical ocular therapeutic for ocular pain, in accordance with the techniques of the present disclosure.

FIG. 4 is an example of an ocular therapeutic of FIG. 3 retained within 0.01-fluid-ounce (fl. oz.) vials.

FIG. 5 is a flowchart depicting a method of using the system of FIG. 3 to make ocular therapeutic eyedrops for pain.

### DETAILED DESCRIPTION

The cornea is the transparent front layer of the human eyeball. Richly innervated by sensory nerves, this tissue is responsible for bending light to focus it onto the retina. The epithelium is the cell layer lining the corneal surface and is continuous with the remainder of the external tissue of the eye, covering the white sclera and extraocular muscles, termed the conjunctiva. Presently, accidental abrasions and acquired defects to the corneal and conjunctival epithelium are extremely painful, and represent the most common eye-related injuries presenting to emergency rooms, accounting for around 30% of eye-related complaints. The conjunctiva is also intentionally harmed during ocular surgery as a means to access the anterior and posterior intraocular chambers, extraocular muscles, or surrounding tissues. Trauma exists in many forms, including mechanical, chemical, or infectious origins, and may affect the corneal or conjunctival epithelium. In cases of a full-thickness abrasion, or open globe laceration, patients experience exquisite pain. This injury represents the most serious injury in eye care, which can affect the visual prognosis permanently. Infectious causes of pain include conjunctivitis or keratitis, such as from a bacterial infection, or a corneal ulcer, which involves focal bacterial proliferation in addition to an abrasion (an infected scratch of the epithelium). In addition to traumatic and infectious causes of pain, inflammation of the connective tissues of the eyeball, such as the white sclera or its loose covering termed the episclera, are also associated with ocular discomfort.

In some cases, epithelial tissue can be intentionally damaged intraoperatively, i.e., during the course of a surgical procedure, by either mechanical or pharmaceutical mechanisms. For instance, during glaucoma surgery, the surgeon forms an epithelial abrasion through an “anchoring stitch” in order to retain the eyeball in a certain orientation and expose specific portions of the eyeball. Similarly, standard cataract surgeries require the surgeon to incise through the cornea. Further, eye-muscle surgery for “lazy eye” involves intentional incisions into the conjunctival epithelium. In fact, practically any intraocular injection, surgery, or procedure on the cornea or conjunctiva results in collateral trauma to this epithelial tissue and the resulting postoperative patient discomfort, including by such means as the topical ocular disinfectants which themselves cause resulting short-term eye irritation.

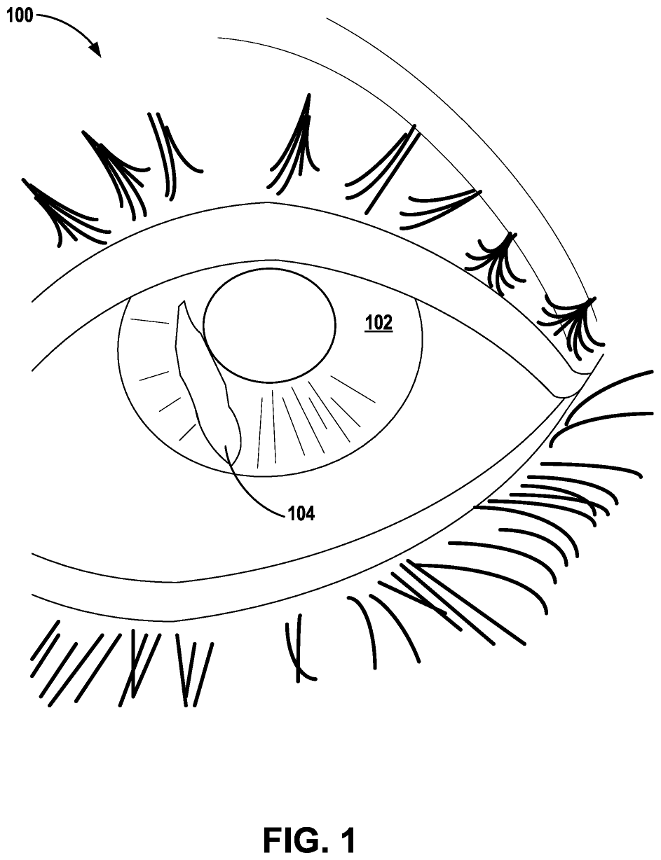

As a visual example, FIG. 1 depicts an eyeball 100 of a patient, in which the epithelial tissue of the cornea 102 features a noticeable abrasion 104. In the example of FIG. 1, the abrasion 104 has been accentuated to facilitate visual diagnosis: a topical fluorescent-yellow dye is topically applied to the eyeball 100, which then fills the abrasion 104. The dye is then illuminated with a cobalt-blue-filtered light, which clearly resolves the abrasion 104 as a bright-green region of the cornea 102. These can also be diagnosed on the conjunctival epithelium (not pictured).

The fluorescent dye is often combined with a local anesthetic, most commonly, proparacaine hydrochloride (available generically, or under the brand name Alcaine® from Alcon AG, headquartered in Geneva, Switzerland). A single drop of this topical mixture provides the patient immediate pain relief during diagnosis of the abrasion 104, lasting about 15 to 30 minutes, on average.

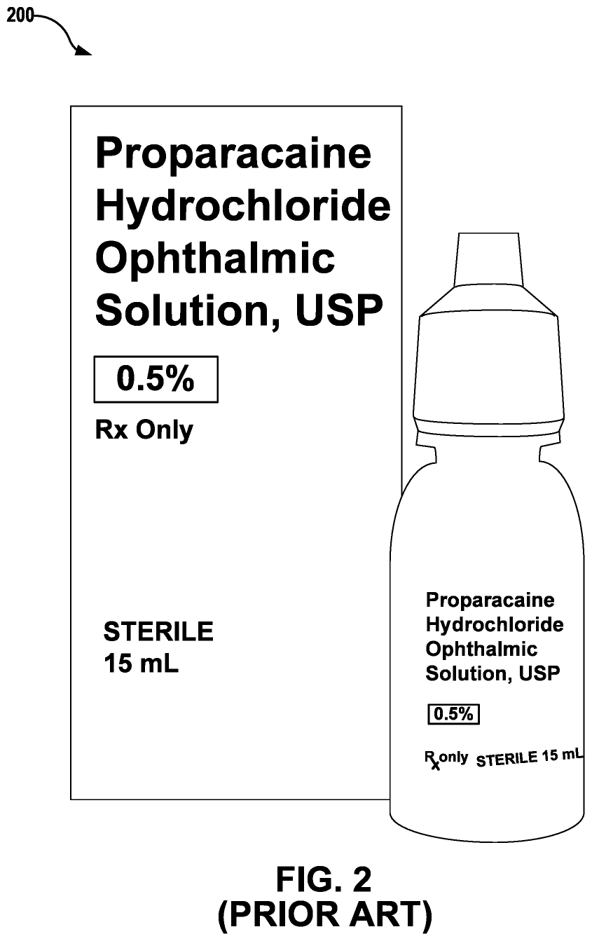

FIG. 2 depicts an example of a vial of generic proparacaine solution 200, available only by prescription. Standard ophthalmic and optometric practice strongly discourages emergency medical personnel or ophthalmologists from prescribing any additional proparacaine to the patient, whose pain quickly returns post-diagnosis, and often lasts for hours-to-days thereafter. The prevailing concern is that the patient could inadvertently apply too much of the anesthetic, causing corneal toxicity that would actually exacerbate their condition. Case reports exist of anesthetic medication overuse, which if continuously applied for several weeks, could cause a painful ulcer and severely impair vision.

Instead, the current standard of care for an ocular epithelial abrasion 104 varies widely. One option for medical providers is to prescribe an antibiotic to prevent infection, and additional artificial tears for lubrication and to simulate reflexive tearing, while allowing the abrasion 104 to heal naturally over time. One of the most commonly prescribed medications for this affliction is an Erythromycin ointment. The standard package insert included with Erythromycin states that it is indicated for “superficial ocular infections involving the conjunctiva and/or cornea”—notably absent is any reference at all to epithelial abrasions (e.g., preemptive infection prevention for damaged tissue), let alone the treatment of associated pain. While eye “patching” for abrasions was historically considered useful to reduce pain by diminishing eyelid movement against the ocular surface, this practice has fallen out of favor due to the risks of bacterial proliferation beneath the patch. Thus, the standard of care does not include a rapid-acting topical therapeutic to address patient discomfort while the epithelium heals. These observations emphasize how viable options for patient-administered pain relief in trauma are not being pursued with any sense of urgency.

Accordingly, there is an overwhelming need in the field of ophthalmology to provide a topical anesthetic that simultaneously addresses both of these diametrically opposed concerns, i.e., that is (1) sufficiently concentrated to alleviate the patient's pain, but (2) dilute enough to negate the risk of corneal toxicity when self-administered by the patient.

The present disclosure describes exemplary systems, devices, and techniques for treating pain from a corneal or conjunctival epithelial abrasion, whether accidental or post-operative, and pain from full-thickness eye lacerations or other mechanisms such as eye dryness, or inflammation of the eye ball (episcleritis or scleritis). In particular, the topical treatments described herein involve an analgesic (preferably, proparacaine hydrochloride) combined with an antibiotic (such as moxifloxacin, or the equivalent), mixed at a predetermined preferred ratio (e.g., within a substantially narrow range of ratios) so as to strike a balance between the competing interests detailed above.

While certain ophthalmic treatments have been devised in the past, none have sufficiently addressed this particular trauma, and patients remain generally dissatisfied. For instance, U.S. Pat. No. 6,716,830, assigned to pharmaceutical company Alcon/Novartis, describes various combinations of moxifloxacin with other medications. However, the specific application of corneal or conjunctival abrasions is not disclosed, nor is the combination of moxifloxacin and proparacaine, let alone in the breakthrough ratio range described herein.

U.S. Pat. No. 5,760,077 to Lee Shahinian, Jr. describes an analgesic solution of dilute anesthetic “administered to a patient suffering from corneal epithelial defect.” While this reference includes a passing reference to combinations that include antibiotics and several other agents, Shahinian fails to elaborate any further on which antibiotics, let alone on the preferred relative concentrations thereof, or addressing the discomfort from defects of the ocular surface anywhere outside of the cornea, specifically.

U.S. Pat. No. 8,414,912 to Ciolino et al., and U.S. Pat. No. 10,555,804 to de Juan, Jr. et al., both describe medication-infused (e.g., “drug-eluting”) contact-lens-type devices configured to fit over the cornea in cases of trauma. Such applications fall firmly outside the scope of the present disclosure. The user-friendly formulations described herein are intended to be safely and conveniently self-administrable by the patient as-needed, i.e., without presenting potential sources of further irritation to the cornea.

In contrast to the previous examples, the scope of the present disclosure is limited to a formulation of eye medication in a topical liquid or semi-viscous gel solution for treating ocular pain, such as an ocular epithelial abrasion, for example, whether accidental or post-operative. In particular, the topical treatments described herein consist of a topical anesthetic/analgesic (preferably, proparacaine hydrochloride, which has safety data in dilute formulations, although other anesthetics have been studied and are reasonably viable) combined with an antibiotic (such as moxifloxacin, or the functional equivalent), mixed together according to a preferred volumetric ratio (e.g., within a substantially narrow range of volumetric ratios) that achieves a unique balance between the competing interests of efficacy and patient safety. Other examples of suitable anesthetics include tetracaine, cocaine, chloroprocaine, procaine, hexylcaine, bupivacaine, lidocaine, benoxinate, mepivacaine, prilocaine, and etidocaine. Suitable classes of antibiotics include fluoroquinolones (such as moxifloxacin), cephalosporins, polymyxins, aminoglycosides, and sulfonamides.

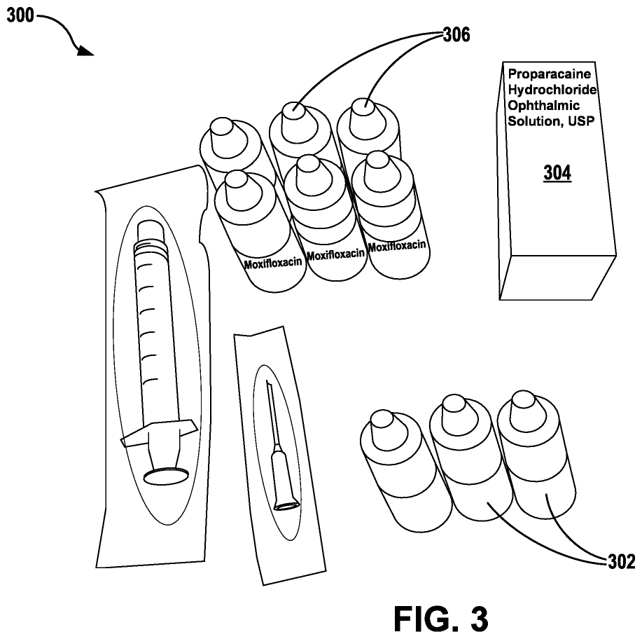

FIG. 3 depicts an example system 300 for making topical ocular therapeutic eyedrops 302, in accordance with the techniques of the present disclosure. To ensure the eye heals without the risk of bacterial proliferation, the eyedrop 302 is a mixture of a topical anesthetic 304 and an antibiotic 306. In the example depicted in FIG. 3, the anesthetic 304 includes proparacaine hydrochloride 0.5% (e.g., proparacaine 200 of FIG. 2). That is, 0.5% (about 5 milligrams (mg) per milliliter (mL)) of the anesthetic 304 is active proparacaine hydrocholoride, and 99.5% of the anesthetic 304 is inactive ingredients, such as glycerin and sterile water.

In an ointment formulation of topical drug delivery, whereby an inert natural or synthetic gel or ointment such as cellulose composes approximately 95-99%, a higher viscosity medium leads to prolongation of the topical treatment on the ocular surface and, in effect, may increase both pain relief and antibiotic effectiveness.

The antibiotic 306 includes moxifloxacin 0.5%, an FDA-approved fluoroquinolone class commonly prescribed in contact-lens wearers, for larger abrasions, or for central-involving abrasions. That is, 0.5% (about 5 mg/mL) of the antibiotic 306 is active moxifloxacin hydrochloride, and 99.5% of the antibiotic 306 is inactive ingredients (or “excipients”), such as boric acid, sodium chloride, and purified water (and in some cases, hydrochloric acid and/or sodium hydroxide to adjust pH).

Tests have revealed substantial benefits when the antibiotic 306 and the anesthetic 304 are combined in a volumetric ratio of about 5:1 to about 15:1 of antibiotic-to-anesthetic, preferably from about 9:1 to about 11:1. In the example depicted in FIG. 3, the medicated eyedrops 302 include a ratio of about 9:1 or about 10:1 of antibiotic 306 to anesthetic 304. As a few illustrative examples: In the case of a 5:1 ratio, the resulting mixture 302 consists of about 0.083% active anesthetic 304, about 0.417% active antibiotic 306, and about 99.5% inactive excipients. In the case of a 9:1 ratio, the resulting mixture 302 consists of about 0.05% active anesthetic 304, about 0.45% active antibiotic 306, and about 99.5% inactive excipients. In the case of a 10:1 ratio, the resulting mixture 302 consists of about 0.045% active anesthetic 304, about 0.455% active antibiotic 306, and about 99.5% inactive excipients. In the case of an 11:1 ratio, the resulting mixture 302 consists of about 0.0417% active anesthetic 304, about 0.4583% active antibiotic 306, and about 99.5% inactive excipients. In the case of a 15:1 ratio, the resulting mixture 302 consists of about 0.03125% active anesthetic 304, about 0.46875% active antibiotic 306, and about 99.5% inactive excipients. Accordingly, anesthetic 304 can range from about 0.030% to about 0.085%; antibiotic 306 can range from about 0.415% to about 0.470%; and the inactive excipients can range from about 99.3% to about 99.7%.

Within this approximate range of concentration ratios, the combined eyedrops 302 adequately treat pain without compromising corneal nerve sensation to an undesirable degree. The test subjects retained some corneal sensation, with a mild anesthetic effect lasting up to an hour or more. The compounded medication 302 can be safely re-applied, as needed, to alleviate patients' pain while their corneal tissue heals over the next several hours-to-days. The medication 302 is found to be sufficiently well-tolerated, and presents no clinically significant side effects. Accordingly, by using an antibiotic and excipient to dilute the anesthetic at a ratio of about 9:1 or 10:1, a patient's pain is well-controlled with the topical formulation, and a distinct advantage is expected to result in the form of substantially increased patient-compliance, and therefore, improved clinical outcomes. Even further, it is asserted that the formulations detailed in this disclosure can help combat the American opioid epidemic. That is, by a well-tolerated, rapid-acting and self-administered topical route to alleviate their source of pain, patients are not motivated to pursue alternative, more addictive and systemic solutions.

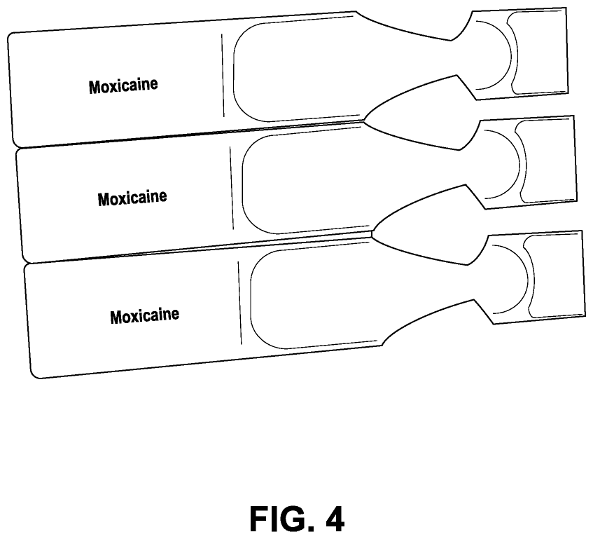

In preferred examples, the medicated eyedrops 302 could be packaged and distributed within standard-size 0.01-fluid-ounce (fl. oz.) vials, such as those depicted in FIG. 4, and as commonly used for non-prescription, preservative-free artificial tears. These smaller-sized doses further help alleviate concerns of toxicity otherwise associated with proparacaine hydrocholoride, i.e., prior to its dilution with the antibiotic and excipient. It may also be packaged similar to standard eye drop volumes, such as 2-5 mL, or thereabouts.

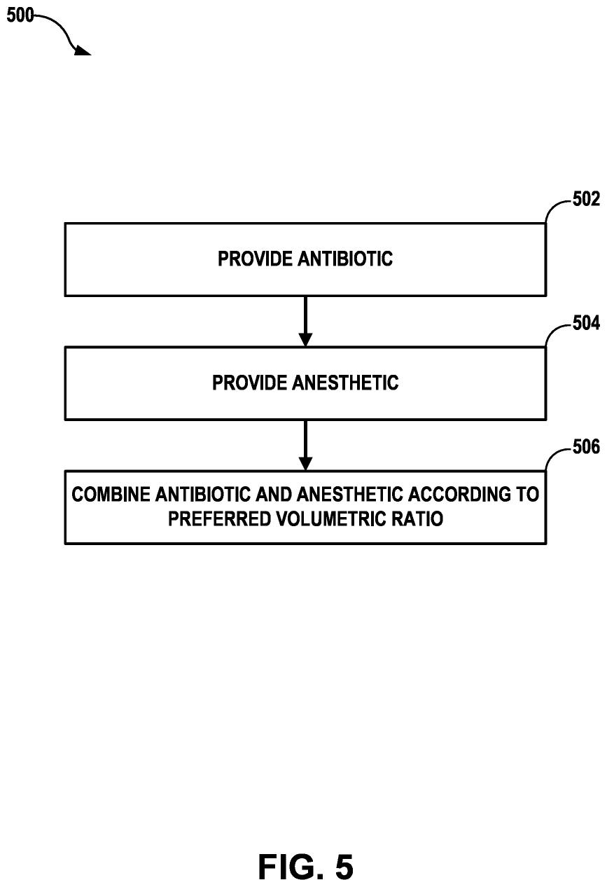

FIG. 5 is a flowchart depicting a method of using the system 300 of FIG. 3 to make topical ocular therapeutic eyedrops 302. The method involves first providing an antibiotic 306 (Step 502) and a topical anesthetic 304 (Step 504). The method further involves using the antibiotic 306 and excipient to substantially dilute the anesthetic 304, by a factor of about 10 (Step 506). For instance when the anesthetic 304 includes 0.5% proparacaine hydrochloride, the antibiotic 306 and excipient are used to dilute the combined solution down to about 0.05% proparacaine hydrochloride. In some cases, a preservative can also be added to the inactive portion, such as benzalkonium chloride 0.01% (or the functional equivalent). The resulting medication can then be packaged and distributed as medicated eyedrops 302 for use in alleviating short-term ocular pain, such as that associated with an ocular epithelial abrasion as it heals.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention, which is to be given the full breadth of the appended claims and any functional equivalents thereof.

What is claimed is:

1. A method of forming a topical medication for treating ocular pain or discomfort, the method comprising:
providing a topical antibiotic;
providing a topical anesthetic;
providing an excipient, and
combining the antibiotic, the anesthetic, and the excipient according to a predetermined volumetric ratio of the antibiotic and the excipient relative to the anesthetic, wherein the volumetric ratio is between about 5:1 and about 15:1.

2. The method of claim 1, wherein the anesthetic comprises proparacaine hydrochloride at a concentration of about 0.5%, prior to combining the antibiotic and the anesthetic.

3. The method of claim 1, wherein the antibiotic comprises a fluoroquinolone-class antibiotic.

4. The method of claim 3, wherein the fluoroquinolone-class antibiotic comprises moxifloxacin at a concentration of about 0.5%, prior to combining the antibiotic, the anesthetic, and the excipient.

5. The method of claim 1, wherein the volumetric ratio is between about 9:1 and about 11:1.

6. A topical medication for treating ocular pain or discomfort, the topical medication comprising:
an antibiotic defining a first concentration of about 0.415% to about 0.470% of the topical medication;
a topical anesthetic defining a second concentration of about 0.030% to about 0.085% of the topical medication; and
an excipient defining a third concentration of about 99.3% to about 99.7% of the topical medication.

7. The topical medication of claim 6, wherein the anesthetic comprises proparacaine, tetracaine, cocaine, chloroprocaine, procaine, hexylcaine, bupivacaine, lidocaine, benoxinate, mepivacaine, prilocaine, or etidocaine.

8. The topical medication of claim 7, wherein the anesthetic comprises proparacaine hydrochloride.

9. The topical medication of claim 6, wherein the antibiotic comprises fluoroquinolone, cephalosporin, polymyxin, aminoglycoside, or a sulfonamide-class antibiotic.

10. The topical medication of claim 6, wherein the excipient comprises an inert polymer, or a gel, an ointment, a cream, a suspension, a solution, or a sustained-release vehicle.

11. The topical medication of claim 6, wherein the topical medication is filled and packaged in a single-use or a multiple-dose container, dispenser, or reservoir.

12. The topical medication of claim 6, wherein the first concentration is about 0.4500% to about 0.4583%, and wherein the second concentration is about 0.0417% to about 0.0500%.

13. The topical medication of claim 6, wherein the inactive portion comprises a preservative.

14. A method of treating an ocular pain or discomfort of a patient, the method comprising:
dispensing a topical medication from a reservoir, wherein the topical medication comprises:
an antibiotic defining a first concentration of about 0.415% to about 0.470% of the topical medication;
a topical anesthetic defining a second concentration of about 0.030% to about 0.085% of the topical medication; and
an excipient defining a third concentration of about 99.3% to about 99.7% of the medication; and
applying the medication topically to an ocular surface of the patient.

15. The method of claim 14, wherein the topical anesthetic comprises proparacaine, tetracaine, cocaine, chloroprocaine, procaine, hexylcaine, bupivacaine, or lidocaine.

16. The method of claim 15, wherein the topical anesthetic comprises proparacaine hydrochloride.

17. The method of claim 14, wherein the antibiotic comprises a fluoroquinolone-class antibiotic, a cephalosporin-class antibiotic, a polymyxin-class antibiotic, an aminoglycoside-class antibiotic, or a sulfonamide-class antibiotic.

18. The method of claim 14, wherein the fluoroquinolone-class antibiotic comprises moxifloxacin.

19. The method of claim 14, wherein the reservoir comprises a 0.01-fluid-ounce vial or a 2-to-5 milliliter (mL) bottle.

20. The method of claim 14, wherein the excipient comprises a gel.
